(19) United States
(12) Patent Application Publication
(10) Pub. No.: US 2008/0107121 A1
(43) Pub. Date: May 8, 2008

(54) Network roaming and reporting system of physiological parameters and method thereof

(75) Inventors: Shuenn-Tsong Young, Taipei City (TW); Chung-Wang Lee, Hsinchu (TW); Chao-Hung Lin, Taipei City (TW); Hao-Chieh Jao, Taipei City (TW)

Correspondence Address: BACON & THOMAS, PLLC, 625 SLATERS LANE, FOURTH FLOOR, ALEXANDRIA, VA, 22314, US

(73) Assignee: National Yang-Ming University, Taipei City (TW)

(21) Appl. No.: 11/593,530

(22) Filed: Nov. 7, 2006

(51) Int. Cl. H04L 12/66; A61B 5/00; H04Q 7/00

(52) U.S. Cl. 370/401; 600/300; 370/310

(57) ABSTRACT

The present invention discloses a network roaming and reporting system of physiological parameters and its method. The system includes at least one wireless transmission module, at least two gateways, an Internet and a server. The wireless transmission module is connected to a portable measuring instrument for receiving a user's physiological parameters measured by the measuring instrument and using an identification code of the wireless transmission module to encode the physiological parameters into a packet. The gateway is provided for receiving the packet from the wireless transmission module and analyzing and processing the packet based on a user's physiological parameter history data or threshold to determine whether or not the user's life condition is critical and automatically generating a precaution reporting signal to report to a related family member through Internet or telephone network if the life condition of the user is critical.

### FIELD OF THE INVENTION

The present invention relates to a network roaming and reporting system of physiological parameters and its method, and more particularly to a system and a method capable of reporting to a patient's related contact person through Internet if the patient's life condition is critical.

### BACKGROUND OF THE INVENTION

As the world's average fertility rate drops gradually, the population ageing issue catches more attentions, and the problems caused by the elderly population become increasingly serious, and thus the demand for elderly care and remote homecare increases constantly. In recent years, information technology keeps advancing, the applications of remote homecare and medical services become more diversified, and many developed countries aggressively conduct researches and promotions on remote homecare and medical services to solve the problems caused by the elderly population. Such arrangement not only helps medical institutes including hospitals or clinics to achieve the purposes of sharing resources and saving costs, but also effectively promoting homecare and medical services to remote districts with insufficient medical resources.

In general, the so-called “remote homecare” or “remote medical service” connects a patient and a family, or a patient and a doctor at two different places and transmits information such as a patient's physiological parameters (including blood pressure, heartbeat or blood sugar level) to a family or a doctor through Internet or telephone network technology, so as to compile and save the information as the patient's physiological parameter history data and determine the patient's condition. If a patient feels unwell or a patient's life condition is critical, timely homecare or medical services will be provided. Thus, remote homecare or remote medical services can overcome the limitations of time and space by transferring the medical activities originally held at a medical hospital or a clinic to the patient's home. To build a good remote homecare or remote medical service system, the following three conditions must be met:

(1) Data Computerization: Various kinds of medical treatment information must be fully computerized; for instance, information such as texts, graphs, static images and video voices must be computerized, so that the information and data can be transmitted rapidly through Internet.

(2) Software System: It is necessary to have a set of software capable of integrating texts, data, graphs, images, voices and videoconferencing contents to quickly access the required data between a patient and a doctor at different locations and successfully provide remote homecare or remote medical services. In the past, the remote homecare or remote medical treatment primarily transmitted images through one-way and two-way image transmissions. However, the present remote homecare or medical treatment emphasizes on transmitting a patient's physiological parameters (including blood pressure, heartbeat or blood sugar level) or medical images (such as X-ray films or pathology slides), or simultaneously conducts two-way transmission of images and voice messages, so that a doctor can perform a remote diagnosis directly at the system. For instance, a doctor can simultaneously transmit and inquire information of a patient's condition through sounds and images and perform a medical checkup (including electrocardiogram, auscultation or blood sugar monitoring) for the patient based on the physiological information such as texts, data or graphs measured by the patient's own measuring instrument (including an electrocardiograph, a sphygmomanometer or a blood sugar meter).

(3) Telecommunication Network: It is necessary to build a fast wideband telecommunication network (such as Internet or telephone network) for transmitting a patient's various medical treatment information quickly to a doctor or a family at a remote end, so that the doctor or family can immediately identify the patient's current condition and provide appropriate help.

In the foregoing three major conditions, the invested research funds and manpower for computerizing various different medical treatment information and building a remote homecare or remote medical service software capable of integrating texts, data, graphs, images, voices and video contents must be huge, except the fast wideband telecommunication network tends to become lower and more popular. As a result, the costs incurred for the applications of a remote homecare or remote medical service system remains very high and unaffordable to the general public. What is more, the traditional remote homecare or medical service systems still have the following two major drawbacks. Firstly, these systems are built at a fixed residence or office of a patient and a doctor (or a patient and a family) at different locations, such that if a patient leaves his/her fixed residence or office, the systems cannot monitor the patient's physiological conditions anymore, and thus creating a tremendous psychological burden to the patient, and the patient dares not to leave the residence or office (similar to taking the patient into custody). Such result definitely will adversely affect the patient's health, particularly to a patient with a chronic disease. Secondly, the physiological parameters obtained by monitoring the patient's physiological condition by these systems are transmitted and saved into a specific doctor's office or a specific medical hospital or clinic. Once if the patient falls ill in other districts, it is quite common that the personal related physiological parameter history data cannot be obtained immediately and thus causing a delay of medical treatment or even an unredeemable regret.

Therefore, finding a way of using the existing low-price wideband network, low equipment cost and simple system architecture to design a remote homecare and medical service system to achieve the remote homecare or remote medical service effect, such that patients can freely travel to other places without worrying about being unable to have proper medical care and treatments, and allow users to have a full control and decision-making power on their own physiological parameter history data is an important research subject for manufacturers of the related industry.

### SUMMARY OF THE INVENTION

In view of the foregoing shortcomings of the prior art, the inventor of the present invention based on years of experience to conduct extensive researches and experiments and finally invented a network roaming and reporting system of physiological parameters and method thereof, in hope of acquiring a user's physiological parameter history data collected and saved into the system by the user at regular time through the network roaming mechanism, or acquiring a physiological parameter threshold set by the user, such that when the life condition of the patient is determined to be critical, a precaution reporting mechanism will be started to report the patient's condition immediately to a related department or a family member, so as to achieve the remote homecare and medical services without being limited by geographical districts.

Therefore, it is an objective of the present invention to overcome the foregoing shortcomings by providing a system comprising at least one wireless transmission module, at least two gateways, an Internet and a server, wherein the wireless transmission module is connected to a portable measuring instrument (such as a sphygmomanometer or a blood sugar meter) for receiving a user's physiological parameters (including blood pressure, heartbeat or blood sugar level) measured by the measuring instrument, and using an identification code of the wireless transmission module to encode the physiological parameters into a first packet which is transmitted by a wireless method. The gateway is provided for receiving the first packet transmitted from the wireless transmission module, and analyzing and processing the first packet based on the user's physiological parameter history data collected and stored at regular time or based on a physiological parameter threshold set by a user, and determining whether or not the user's life condition becomes critical and automatically generating a precaution reporting signal, and using an identification code of the wireless transmission module and an exclusive identification code of the gateway to encode the precaution reporting signal into a second packet and transmit the second packet out; and the server achieves a roaming mechanism of physiological parameters between any two gateways at different locations through Internet, such that one gateway can access the user's physiological parameter history data collected and saved into another gateway at regular time, or can access a physiological parameter threshold set by a user from another gateway. If the server receives a precaution reporting signal transmitted from the gateway, the server will base on the contact information (such as email address or telephone number) of the logged-on user's related department or family member to report to the related department or family member through Internet or telephone network. Regardless of the location of the user, the user can acquire proper remote homecare and medical services through the system.

Another objective of the present invention is to provide a system such that if a user issues an emergency signal through the wireless transmission module, the wireless transmission module will use its identification code to encode the emergency signal into a third packet and transmit the third packet out by a wireless method. When the gateway receives the third packet transmitted from the wireless transmission module and determines that the wireless transmission module has produced the emergency signal, the gateway automatically generates a precaution reporting signal, and uses an identification code of the wireless transmission module and an exclusive identification code of the gateway to encode the precaution reporting signal into a fourth packet and transmit the fourth packet out. When the server receives the precaution reporting signal, the server bases on the contact information (such as email address or telephone number) of a logged-on user's related department or family member corresponding to the identification code of the wireless transmission module and the address data of the identification code of the gateway to transmit a correct address of the emergency signal transmitted from the user to report to the related department or family member through Internet or telephone network.

To make it easy for our examiner to understand the objective, shape, structure, characteristics and performance of the present invention, a detailed description of the preferred embodiments with reference to the accompanying drawings are given below.

### BRIEF DESCRIPTION OF THE DRAWINGS

FIG. 1 is a schematic view of a system architecture used within a user environment in accordance with the present invention;

FIG. 2 is a schematic view of a system architecture used in a district beyond a user environment in accordance with the present invention;

FIG. 3 is a flow chart of a gateway processing in accordance with a preferred embodiment of the present invention; and

FIG. 4 is a flow chart of a server processing in accordance with a preferred embodiment as depicted in FIG. 3.

### DETAILED DESCRIPTION OF THE PREFERRED EMBODIMENTS

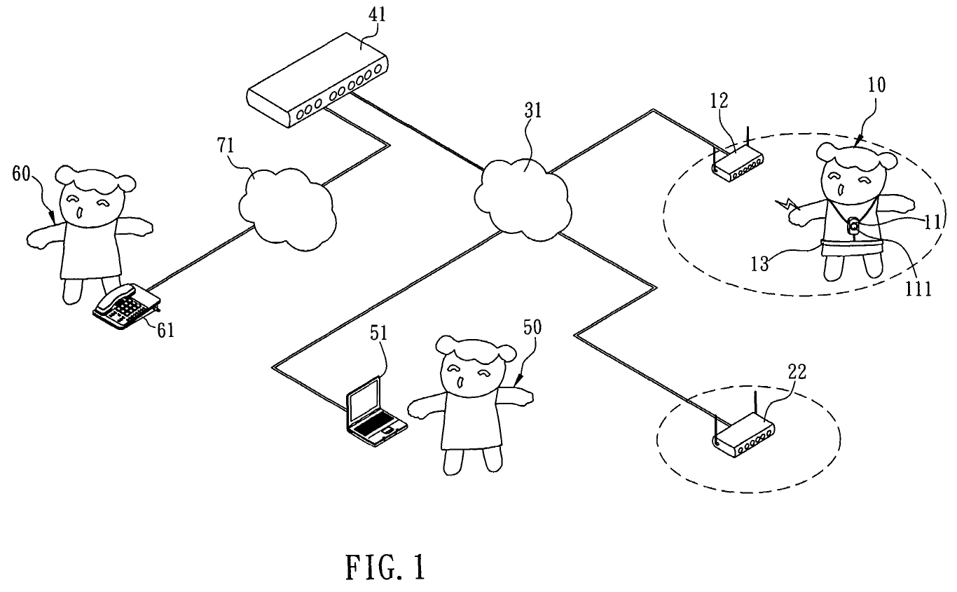

Referring to FIG. 1 for a network roaming and reporting system of physiological parameters and method thereof, the system comprises at least one wireless transmission module 11, at least two gateways 12, 22, an internet 31 and a server 41. The wireless transmission module 11 is connected to a portable measuring instrument 13 (such as a sphygmomanometer or a blood sugar meter) for receiving a user's physiological parameters (including blood pressure, heartbeat or blood sugar level) measured by a measuring instrument 13, and using the identification (ID) code (which is an exclusive personal identification code of a user 10) stored in the wireless transmission module 11 to encode the physiological parameters into a first packet and transmit the first packet out through a wireless method. The gateways 12, 22 are provided separately for receiving surrounding first packets transmitted from the authenticated wireless transmission module. After the first packet is decoded, the physiological parameters are stored into a corresponding anamnesis database of a user 10, according to the different identification codes to compile the physiological parameters collected and saved at regular time into a physiological parameter history data, and analyze and process the received physiological parameters to determine whether or not the life condition of the user 10 is critical. The user 10 also can use a physiological parameter threshold of the gateways 12, 22 for analyzing and processing the received physiological parameters to determine whether or not the life condition of a user 10 is critical. If yes, the gateway 12 will automatically generate a precaution reporting signal and will use an identification code of the wireless transmission module 11 and an exclusive identification code of the gateway 12 to encode the precaution reporting signal into a second packet and transmit the second packet to the server 41 through Internet 31. If the server 41 receives the precaution reporting signal transmitted from the gateway 12, the server 41 will start a precaution reporting mechanism to report to the related department 50 or family member 60 by an email or a voice phone, or a computer 51 or a telephone 61 through Internet 31 or telephone network 71 according to the contact information (such as email address or telephone number) of the related department (such as a hospital or a doctor) 50 or a family member 60 of the corresponding logged-on user 10 based on the identification code of the wireless transmission module 11 its management database.

Referring to FIG. 1, the wireless transmission module 11 further installs an emergency button 111 or a built-in a sensor (not shown in the figure), wherein the emergency button 111 is provided for the user 10 to press in the case of an emergency, and the wireless transmission module 11 will produce an emergency signal, and the sensor is provided for detecting any abnormal movement (such as the patient falling down quickly and lying for a long time) of the user 10, such that the wireless transmission module 11 will produce an emergency signal, and the wireless transmission module 11 will encode the emergency signal into a third packet by an identification code and transmit the third packet out by a wireless transmission method. After the gateway 12 receives the third packet and determines that the wireless transmission module 11 has produced the emergency signal, the gateway 12 will automatically transmit a precaution reporting signal to the server 41 through the Internet 31. If the server 41 receives the precaution reporting signal, the server 41 will base on the contact information (such as email address or telephone number) of a related department 50 or a family member 60 of a logged-on user 10 corresponding to an address data of the identification code of the gateway 12 to send a correct address of the emergency signal transmitted by the user 10 to report to the related department 50 or family member 60 through Internet 31 or telephone network 71.

It is noteworthy that when a user 10 uses the system of the present invention for the first time, the user 10 needs to register the following data into the management database of the server 41:

(1) The identification code of the wireless transmission module: It is an exclusive identification code of the wireless transmission module 11, which is also an exclusive personal identification code of the user 10;

(2) The contact information (such as a telephone number or an email address), and the basic information and related contact person (such as a family member 60, a doctor or a medical hospital or clinic) of the user 10: These data correspond to an identification code of the wireless transmission module 11 for facilitating the server 41 to identify the user 10 and related contact person based on the identification code of the wireless transmission module 11 carried in the received first packet;

(3) The identification code of the gateway: It is an exclusive identification code of the gateway 12; and

(4) The installation location of the gateway and related contact information (which refers to the contact person's location or the telephone number or email address of an nearby medical hospital or clinic): These data correspond to the identification code of the gateway 12 for facilitating the server 41 to identify the installation location of the gateway 12 and the related contact person based on the identification code of the gateway 12 carried in the received first packet.

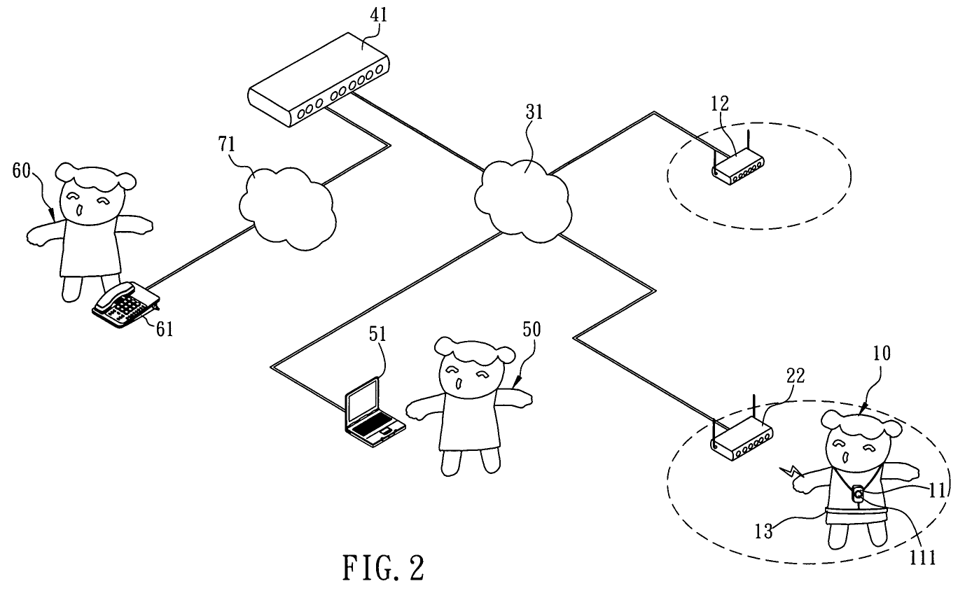

In the present invention, the server 41 connects the gateways 12, 22 installed at different districts through the Internet 31 to achieve the roaming mechanism of physiological parameters, such that if a user 10 goes out or stays in a friend's place, and the friend's place also has a gateway 22 as shown in FIG. 2, and the gateway 22 is set to permit the wireless transmission module 11 carried by the user 10 to be connected to the server 41 through the gateway 22, then the server 41 will base on the identification code of the wireless transmission module 11 to read the physiological parameter history data of the user 10 collected and saved into the gateway 12 at regular time from home, or read the physiological parameter threshold set by the user 10, and download the history data or threshold to the gateway 22 at the friend's home, after the server 41 and the wireless transmission module 11 are connected. Therefore, the gateway 22 at the friend's place can analyze and process the transmitted physiological parameters based on the wireless transmission module 11 to determine whether or not the life condition of the user 10 is critical or an emergency signal has been transmitted. Regardless of the location of the user 10, the system of the present invention can start a precaution reporting mechanism to report to the user's related department 50 or family member 60 when the user's life condition is critical. It is noteworthy to point out that the gateway 22 at a friend's place keeps receiving the physiological parameters transmitted from the wireless transmission module 11, and a gateway 12 at the user's home also can synchronously or periodically upload the received physiological parameters to the server 41 through the roaming mechanism, so as to synchronously or periodically update the stored physiological parameter history data of the user 10.

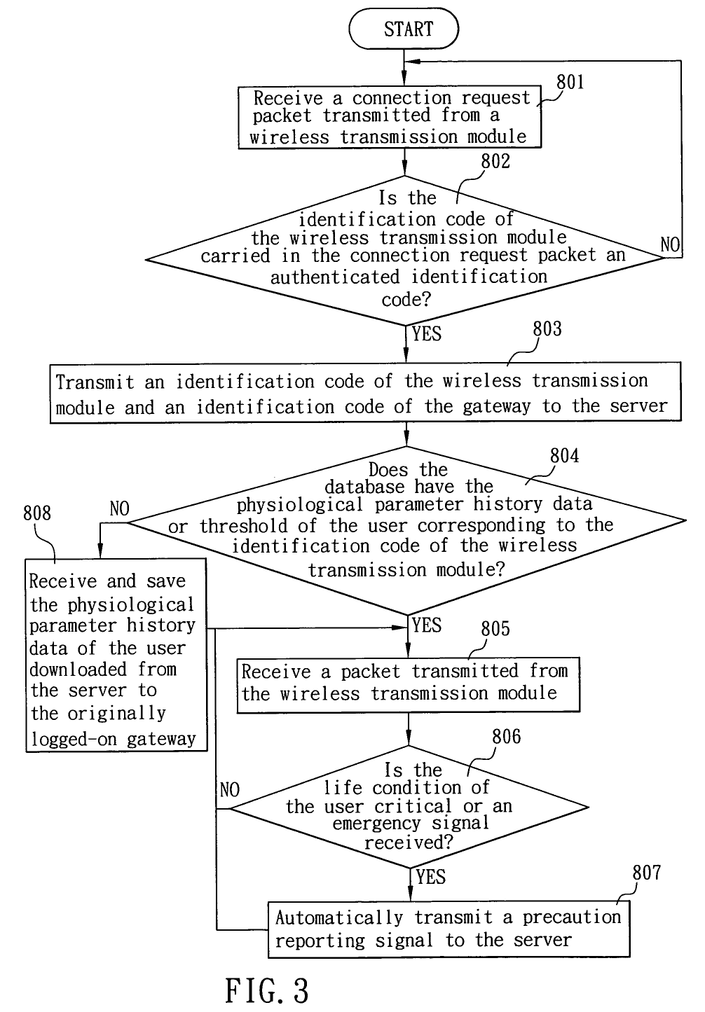

Referring to FIGS. 2 and 3 for a preferred embodiment of the present invention, the design concept and operation of any one of the gateways 12, 22 and the server 41 are described as follows:

If the gateway 22 receives a connection request packet transmitted from a wireless transmission module 11 at the present environment, the control and processing are performed according to the following procedure as shown in FIG. 3:

Step (801): Receive a connection request packet transmitted from a wireless transmission module 11 at the present environment;

Step (802): Examine a lookup table stored in a database to determine whether or not the identification code of the wireless transmission module 11 carried in the connection request packet has been registered as an authenticated identification code. If yes, then continue Step (803); or else refuse the connection request of the wireless transmission module 11 and continue Step (801);

Step (803): Transmit an identification code of the wireless transmission module 11 and an identification code of the gateway 22 to the server 41 through Internet 31; after the server 41 has received the identification codes, the server 41 examines a logon data in its management database according to the identification code of the wireless transmission module 11 and determines whether or not the received identification code of the gateway 22 is the same as the identification code of the logged-on gateway 12. If yes, then it indicates that the wireless transmission module 11 is used at the installation location within the district of the originally logged-on environment; if no, then it indicates that the wireless transmission module 11 is used in a district beyond the originally logged-on environment. Now, the server 41 downloads a physiological parameter history data of a user 10 stored in the gateway 12 from the originally logged-on gateway 12 according to the identification code of the originally logged-on gateway 12, or downloads a physiological parameter threshold set by the user 10 in the gateway 12 and transmits and stores the physiological parameter history data and threshold in a currently used gateway 22;

Step (804): Determine whether or not its database has stored the physiological parameter history data or threshold of the user 10 corresponding to the identification code of the wireless transmission module 11; if yes, then continue Step (805), or else continue Step (808);

Step (805): Receive a third packet transmitted from the wireless transmission module 11 and decode the third packet to obtain a carried physiological parameter or emergency signal of the user 10; and

Step (806): Determine whether or not the physiological parameter of the user 10 carried in the received third packet indicates that the life condition of the user 10 or an emergency signal carried in the received third packet is critical according to the physiological parameter history data or threshold of the user 10; if yes, then continue Step (807), or else continue Step (805);

Step (807): Automatically transmit a precaution reporting signal to the server 41 through the Internet 31, and continue Step (805); and

Step (808): Receive the physiological parameter history data or threshold of the user 10 downloaded from the server 41 to the originally logged-on gateway 12, and save the physiological parameter history data or threshold of the user 10, and continue Step (805).

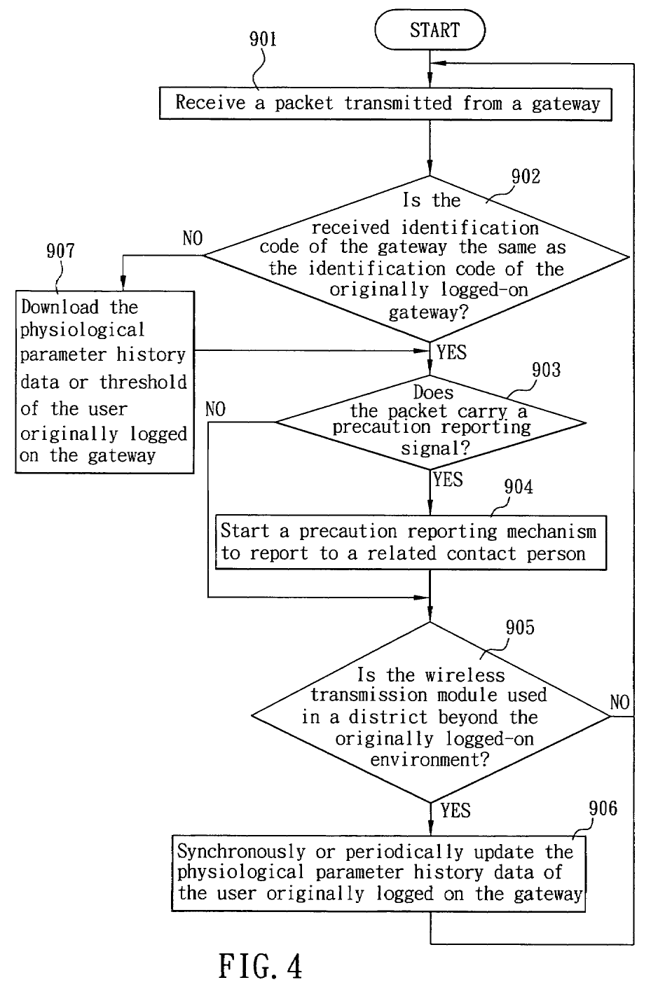

Referring to FIGS. 2 and 4 for a preferred embodiment of the present invention, the following procedure is controlled and processed when the server 41 receives a first packet transmitted from the gateway 22.

Step (901): Receive the first packet transmitted from the gateway 22 and decode the first packet to obtain an identification code of the wireless transmission module 11 and an identification code of the gateway 22;

Step (902): Examine a logon data in its management database, and determine whether or not the received identification code of the gateway 22 is the same as the identification code of the originally logged-on gateway 12 according to the identification code of the wireless transmission module 11; if yes, then it indicates that wireless transmission module 11 is used at an installation location within the district of the originally logged-on gateway 12, and continue Step (903), or else it indicates that the wireless transmission module 11 is used in a district beyond the originally logged-on environment, and continue Step (907);

Step (903): Determine whether or not the second packet carries a precaution reporting signal. If yes, continue Step (904), or else continue Step (905);

Step (904): Start a precaution reporting mechanism, and contact a computer 51 or a telephone 61 of a related department 50 or family member 60 by email or voice phone through the Internet 31 or telephone network 71 according to the contact information (such as email address or telephone number) of the related department 50 or family member 60 of a user 10 in its management database corresponding to the identification code of a logged-on wireless transmission module 11;

Step (905): Determine whether or not the wireless transmission module 11 is used in a district beyond the originally logged-on environment. If yes, then continue Step (906), or else continue Step (901);

Step (906): Synchronously or periodically read a physiological parameter of the user 10 received by the gateway 22, and upload the identification code to the originally logged-on gateway 12 based on the originally logged-on gateway 12, and synchronously or periodically update the stored physiological parameter history data of the user 10, and continue Step (901); and

Step (907): Download the stored physiological parameter history data or threshold of the user 10, and transmit the physiological parameter history data or threshold of the user 10 from the originally logged-on gateway 12 to the currently used gateway 22 according to the identification code of the originally logged-on gateway 12, and continue Step (903).

The present invention adopts a low-cost server 41 and gateways 12, 22 to build a simple structured network platform in the Internet environment, such that the physiological parameter history data or threshold of the user 10 can be roamed between the gateways 12, 22 at different districts, and one of the gateways can access the physiological parameter history data of the user 10 collected and saved at regular time by another gateway or can access the physiological parameter threshold set by the user 10 in another gateway, and determines whether or not the life condition of the user 10 is critical, and transmits a precaution reporting signal, so that the server 41 starts the precaution reporting mechanism to immediately report to the related contact person through the Internet or telephone network based on the data (such as email address or telephone number) of a related contact person of the logged-on user 10. The system of the present invention allows a user 10 to freely travel to places without worrying about being unable to receive proper medical care and treatments. Since the physiological parameter history data or threshold of the user 10 are stored permanently in the gateway at the user's home, therefore the user 10 has absolute control and decision-making power on the history data or threshold to back up, edit and use the history data or threshold to meet the needs for emergencies.

While the invention herein disclosed has been described by means of specific embodiments, numerous modifications and variations could be made thereto by those skilled in the art without departing from the scope and spirit of the invention set forth in the claims.

What is claimed is:

1. A network roaming and reporting system of physiological parameters, comprising:
at least one wireless transmission module, coupled to a portable measuring instrument, for receiving a user's physiological parameters measured by said measuring instrument, and using an exclusive identification code of said wireless transmission module to encode said physiological parameters into a first packet, and transmitting said first packet through a wireless method;
at least two gateways, each for receiving said first packet transmitted from said wireless transmission module, and analyzing and processing said first packet according to a user's physiological parameter history data collected and saved into a database of said gateway at regular time or according to a physiological parameter threshold preset in said database to determine whether or not a user's life condition becomes critical and automatically generate a precaution reporting signal, and encode said precaution reporting signal into a second packet by using an identification code of said wireless transmission module and an exclusive identification code of said gateway, and transmit said second packet out;
an Internet, connected to said each gateway, for receiving said second packet transmitted from said each gateway; and
a server, connected to any two gateways through said Internet, such that one of said gateways accesses said user's physiological parameter history data collected and saved at regular time in said database of another gateway, or accesses said physiological parameter threshold preset in said database of another gateway through said server, and after said server receives said second packet with said precaution reporting signal transmitted from said each gateway and obtains the data of a related contact person of a corresponding user registered in a management database of said server according to said identification code of said wireless transmission module and said exclusive identification code of said gateway, said server reports to said related contact person about the situation relating to said precaution reporting signal through a telecommunication network.

2. The system of claim 1, wherein said management database of said server, comprising:
at least one exclusive identification code of said wireless transmission module;
at least one user's basic information and contact person information corresponding to said exclusive identification code of said each wireless transmission module;
at least two exclusive identification codes of said gateways; and
at least two installation addresses and contact person information of said gateways respectively corresponding to said exclusive identification code of said each gateway.

3. The system of claim 2, wherein said wireless transmission module further comprises an emergency button provided for a user to press during an emergency, such that said wireless transmission module produces an emergency signal accordingly, and uses said identification code of said wireless transmission module to encode said emergency signal into a third packet and transmit said third packet out through said wireless method.

4. The system of claim 2, wherein said wireless transmission module further comprises a built-in sensor, for generating an emergency signal when detecting a user's abnormal movement and using said identification code of said wireless transmission module to encode said emergency signal into a third packet and transmit said third packet out through said wireless method.

5. The system of claims 3, wherein said gateway automatically generates said precaution reporting signal when receiving said third packet transmitted from said wireless transmission module and determining that said wireless transmission module has generated said emergency signal, encodes said precaution reporting signal into a fourth packet by using said identification code of said wireless transmission module and said exclusive identification code of said gateway, and then transmits said fourth packet out.

6. The system of claims 4, wherein said gateway automatically generates said precaution reporting signal when receiving said third packet transmitted from said wireless transmission module and determining that said wireless transmission module has generated said emergency signal, encodes said precaution reporting signal into a fourth packet by using said identification code of said wireless transmission module and said exclusive identification code of said gateway, and then transmits said fourth packet out.

7. The system of claim 5, wherein said telecommunication network is an Internet.

8. The system of claim 6, wherein said telecommunication network is an Internet.

9. The system of claim 5, wherein said telecommunication network is a telephone network.

10. The system of claim 6, wherein said telecommunication network is a telephone network.

11. A network roaming and reporting system of physiological parameters, which is implemented to a gateway, comprising:
receiving a connection request packet transmitted by a wireless transmission module at the present environment;
examining a lookup table stored in a database of said gateway, and determining whether or not an exclusive identification code of said wireless transmission module carried in said connection request packet has been registered as an authenticated identification code; if yes, then transmitting said exclusive identification code of said wireless transmission module and an exclusive identification code of said gateway to a server through Internet;
determining whether or not a user's physiological parameter history data or threshold corresponding to said identification code of said wireless transmission module is saved in said database; if yes, then receiving said first packet transmitted from said wireless transmission module and decoding said first packet to obtain user's physiological parameters or an emergency signal carried in said first packet; and
determining whether or not said received user's physiological parameters carried in said first packet stands for a critical condition of said user or said received packet carries said emergency signal according to said user's physiological parameter history data or threshold stored in said database; if yes, then automatically generating a precaution reporting signal, and using said exclusive identification code of said wireless transmission module and said exclusive identification code of said gateway to encode said precaution reporting signal into a first packet and transmit said first packet to said sever through Internet.

12. The method of claim 11 wherein, when determining that said user's physiological parameter history data or threshold corresponding to said identification code of said wireless transmission module is not stored in said database, further comprising the step of receiving said user's physiological parameter history data or threshold downloaded by said server from an originally logged gateway.

13. A network roaming and reporting system of physiological parameters, which is implemented to a server, comprising the steps of:
receiving a packet transmitted by a currently used gateway through Internet, and decoding said packet to obtain an identification code of a wireless transmission module and an identification code of said gateway;
examining a logon data stored in a management database of said server, and determining whether or not said identification code of said gateway is the same as an identification code of an originally logged-on gateway based on said identification code of said wireless transmission module; and
if yes, determining whether or not said packet carries a precaution reporting signal; if yes, then starting a precaution reporting mechanism, and reporting to a related contact person through a telecommunication network according to the data of a related contact person of a user registered in said management database corresponding to said identification code of said wireless transmission module.

14. The method of claim 13 wherein, when determining that said received identification code of said currently used gateway is not the same as said identification code of said originally logged-on gateway, further comprising the step of downloading said stored physiological parameter history data or threshold of said user from said originally logged-on gateway to said currently used gateway based on said identification code of said originally logged-on gateway.

15. The method of claims 13 wherein, when determining that said received identification code of said currently used gateway is not the same as said identification code of said originally logged-on gateway, further comprising the step of synchronously or periodically reading said received physiological parameter of said user of said currently used gateway and uploading said received physiological parameter of said user of said currently used gateway to said originally logged-on gateway based on said identification code of said originally logged-on gateway, and synchronously or periodically updating said user's physiological parameter stored in said originally logged-on gateway.

16. The method of claims 14 wherein, when determining that said received identification code of said currently used gateway is not the same as said identification code of said originally logged-on gateway, further comprising the step of synchronously or periodically reading said received physiological parameter of said user of said currently used gateway and uploading said received physiological parameter of said user of said currently used gateway to said originally logged-on gateway based on said identification code of said originally logged-on gateway, and synchronously or periodically updating said user's physiological parameter stored in said originally logged-on gateway.
